(19) United States
(12) Patent Application Publication
(10) Pub. No.: US 2024/0057978 A1
(43) Pub. Date: Feb. 22, 2024

(54) REDUCING TEMPORAL MOTION ARTIFACTS

(71) Applicant: KONINKLIJKE PHILIPS N.V., EINDHOVEN (NL)

(72) Inventors: LEILI SALEHI, WALTHAM, MA (US); GRZEGORZ ANDRZEJ TOPOREK, CAMBRIDGE, MA (US); AYUSHI SINHA, BALTIMORE, MD (US); ASHISH SATTYAVRAT PANSE, BURLINGTON, MA (US); RAMON QUIDO ERKAMP, SWAMPSCOTT, MA (US)

(21) Appl. No.: 18/267,559

(22) PCT Filed: Dec. 14, 2021
(86) PCT No.: PCT/EP2021/085582

§ 371 Date: Jun. 15, 2023

Related U.S. Application Data
(60) Provisional application No. 63/129,364, filed on Dec. 22, 2020.

(51) Int. Cl. A61B 8/08; A61B 5/00; A61B 5/0205

(52) U.S. Cl. CPC A61B 8/5276; A61B 5/0036; A61B 5/02055; A61B 8/0883

(57) ABSTRACT

A computer-implemented method of reducing temporal motion artifacts in temporal intracardiac sensor data, includes: inputting (S120) temporal intracardiac sensor data (110), into a neural network (130) trained to predict, from the temporal intracardiac sensor data (110), temporal motion data (140, 150) representing the temporal motion artifacts (120); and compensating (S130) for the temporal motion artifacts (120) in the received 5 temporal intracardiac sensor data (110) based on the predicted temporal motion data (140, 150).

### TECHNICAL FIELD

The present disclosure relates to reducing temporal motion artifacts in temporal intracardiac sensor data. A computer-implemented method, a processing arrangement, a system, and a computer program product, are disclosed.

### BACKGROUND

Intracardiac sensors are used in various medical investigations in the medical field. For example, in an electrophysiology “EP” study, also known as an EP mapping, and as electro-anatomical mapping “EAM”, procedure, an electrical sensor disposed on an intracardiac catheter is used to sense electrical activity within the heart whilst a position sensor disposed on the catheter provides position data. The electrical activity and position data are used to construct a three-dimensional map of the heart's electrical activity. EP studies are used to investigate heart rhythm issues such as arrythmias and determine the most effective course of treatment.

Cardiac ablation is a common procedure for treating arrythmias and involves terminating faulty electrical pathways from sections of the heart. The electrical activity map provided by an EP study is often used to locate the arrythmia and thus determine the optimal position to perform the ablation. The EP study may be performed a-priori, or contemporaneously with treatment. Arrythmias are treated by creating transmural lesions at identified sources of arrythmia using radiofrequency “RF” ablation, microwave ablation “MV” or cryoablation, or, more recently, irreversible electroporation, in order to isolate them from the rest of the myocardial tissues. During a cardiac ablation procedure, other types of intracardiac sensors may also be used to measure parameters relating to the treatment. For example, a temperature sensor may be disposed on an ablation catheter and used to measure the temperature of the cardiac wall. The ablation catheter may also include a voltage sensor and/or a current sensor, or an impedance measurement circuit for measuring a state of a tissue within the heart such as lesion quality. Similarly, a force sensor may be included on the ablation catheter and used to measure a contact force between a cardiac probe and the cardiac wall.

Other types of intracardiac sensors may also be used during EP studies, cardiac ablation procedures, and other intracardiac procedures, including a blood flow sensor, a microphone, a temperature sensor to measure the temperature of blood, and so forth.

Intracardiac sensors often suffer from temporal motion artifacts which degrade the accuracy of their measurements. For example, cardiac motion and/or respiratory motion degrade the accuracy of data from an intracardiac position sensor that is used in constructing a three-dimensional map of the heart's electrical activity during an EP study.

Conventional approaches to reducing such temporal motion artifacts include the use of filters to suppress artifacts arising from cardiac and respiratory motion. However, filters may add a delay to the signal processing chain. Cardiac and respiration rates may also change during a procedure. The use of filters with constant coefficients may result in the incomplete removal of unwanted frequencies from the sensor measurements. The use of conventional adaptive filters such as a Kalman filter may also give rise to unpredictable behavior in response to sudden changes in sensor input, for example when a sensor's position is changed.

Consequently, there remains a need to reduce temporal motion artifacts in temporal intracardiac sensor data.

### SUMMARY

According to one aspect of the present disclosure a computer-implemented method of reducing temporal motion artifacts in temporal intracardiac sensor data, is provided. The method includes:
receiving temporal intracardiac sensor data, the temporal intracardiac sensor data including temporal motion artifacts;
inputting the temporal intracardiac sensor data, into a neural network trained to predict, from the temporal intracardiac sensor data, temporal motion data representing the temporal motion artifacts; and
compensating for the temporal motion artifacts in the received temporal intracardiac sensor data based on the predicted temporal motion data.

According to another aspect of the present disclosure, a computer-implemented method of providing a neural network for predicting temporal motion data representing temporal motion artifacts from temporal intracardiac sensor data, is provided. The method includes:
receiving temporal intracardiac sensor training data, the temporal intracardiac sensor training data including temporal motion artifacts;
receiving ground truth temporal motion data representing the temporal motion artifacts;
inputting the received temporal intracardiac sensor training data, into a neural network, and adjusting parameters of the neural network based on a loss function representing a difference between temporal motion data representing the temporal motion artifacts, predicted by the neural network, and the received ground truth temporal motion data representing the temporal motion artifacts.

Further aspects, features and advantages of the present disclosure will become apparent from the following description of examples, which is made with reference to the accompanying drawings.

### BRIEF DESCRIPTION OF THE DRAWINGS

FIG. 1 is a schematic diagram illustrating two views of an electro-anatomical map of the left atrium of the heart and includes an intracardiac catheter 100.

FIG. 2 illustrates an example of temporal intracardiac sensor data 110 (Force, upper, Impedance, lower) generated by an ablation catheter, and which data includes temporal motion artifacts 120.

FIG. 3 is a flowchart of an example method of reducing temporal motion artifacts in temporal intracardiac sensor data, in accordance with some aspects of the disclosure.

FIG. 4 is a schematic diagram illustrating an example method of reducing temporal motion artifacts in temporal intracardiac sensor data, in accordance with some aspects of the disclosure.

FIG. 5 is a schematic diagram illustrating a first example of a neural network 130 for predicting temporal motion data 140, 150 representing temporal motion artifacts, in accordance with some aspects of the disclosure.

FIG. 6 is a schematic diagram illustrating a second example of a neural network 130 for predicting temporal motion data 140, 150 representing temporal motion artifacts, in accordance with some aspects of the disclosure.

FIG. 7 is flowchart of an example method of training a neural network to predict temporal motion data 140, 150 representing temporal motion artifacts, in accordance with some aspects of the disclosure.

FIG. 8 is a schematic diagram illustrating an example method of training a neural network to predict temporal motion data 140, 150 representing temporal motion artifacts, in accordance with some aspects of the disclosure.

FIG. 9 is a schematic diagram illustrating a third example of a neural network 130 for predicting temporal motion data 140, 150 representing temporal motion artifacts, in accordance with some aspects of the disclosure.

FIG. 10 is a schematic diagram illustrating a fourth example of a neural network 130 for predicting temporal motion data 140, 150 representing temporal motion artifacts, in accordance with some aspects of the disclosure.

### DETAILED DESCRIPTION

Examples of the present disclosure are provided with reference to the following description and the Figures. In this description, for the purposes of explanation, numerous specific details of certain examples are set forth. Reference in the specification to “an example”, “an implementation” or similar language means that a feature, structure, or characteristic described in connection with the example is included in at least that one example. It is also to be appreciated that features described in relation to one example may also be used in another example, and that all features are not necessarily duplicated in each example for the sake of brevity. For instance, features described in relation to a computer-implemented method may be implemented in a processing arrangement, and in a system, and in a computer program product, in a corresponding manner.

In the following description, reference is made to computer implemented methods that involve reducing temporal motion artifacts in temporal intracardiac sensor data. Reference is made to data generated by an example intracardiac position sensor disposed on an intracardiac catheter during an EP mapping procedure. However, it is to be appreciated that examples of the computer implemented methods may be used with other types of intracardiac sensors to a position sensor, and using other types of interventional devices to a catheter, and with data generated from such sensors during intracardiac procedures other than EP mapping. For instance, examples of intracardiac sensors in accordance with the present disclosure include electrical sensors of voltage, current and impedance that measure electrical activity and other parameters relating to the heart, temperature sensors, force sensors, blood flow sensors, and so forth. Such sensors may be disposed on intracardiac interventional devices such as a guidewire, a blood pressure device, a blood flow sensor device, a therapy device such as a cardiac ablation device, and so forth. Examples of intracardiac sensors in accordance with the present disclosure may be used in intracardiac procedures in general, including for example an EP mapping procedure, a cardiac ablation procedure, and so forth.

It is noted that the computer-implemented methods disclosed herein may be provided as a non-transitory computer-readable storage medium including computer-readable instructions stored thereon which, when executed by at least one processor, cause the at least one processor to perform the method. In other words, the computer-implemented methods may be implemented in a computer program product. The computer program product can be provided by dedicated hardware or hardware capable of running the software in association with appropriate software. When provided by a processor, or “processing arrangement”, the functions of the method features can be provided by a single dedicated processor, by a single shared processor, or by a plurality of individual processors, some of which can be shared. The explicit use of the terms “processor” or “controller” should not be interpreted as exclusively referring to hardware capable of running software, and can implicitly include, but is not limited to, digital signal processor “DSP” hardware, read only memory “ROM” for storing software, random access memory “RAM”, a non-volatile storage device, and the like. Furthermore, examples of the present disclosure can take the form of a computer program product accessible from a computer usable storage medium or a computer-readable storage medium, the computer program product providing program code for use by or in connection with a computer or any instruction execution system. For the purposes of this description, a computer-usable storage medium or computer-readable storage medium can be any apparatus that can comprise, store, communicate, propagate, or transport a program for use by or in connection with an instruction execution system, apparatus, or device. The medium can be an electronic, magnetic, optical, electromagnetic, infrared, or semiconductor system or device or propagation medium. Examples of computer-readable media include semiconductor or solid-state memories, magnetic tape, removable computer disks, random access memory “RAM”, read only memory “ROM”, rigid magnetic disks, and optical disks. Current examples of optical disks include compact disk-read only memory “CD-ROM”, optical disk-read/write “CD-R/W”, Blu-Ray™, and DVD.

FIG. 1 is a schematic diagram illustrating two views of an electro-anatomical map of the left atrium of the heart and includes an intracardiac catheter 100. The intracardiac catheter 100 represents a mapping or ablation catheter and may be used to generate an EP map such as that illustrated in FIG. 1. The intracardiac catheter 100 includes an electrical sensor that is used to sense electrical activity within the heart, and a position sensor that generates position data indicating the position of the electrical sensor. The electrical sensor may for example be a voltage sensor, or a current sensor. Together, a voltage sensor and a current sensor may be configured to provide an impedance measurement circuit for making measurements of the cardiac wall and thereby determining a tissue state. The ablation catheter may additionally include sensors such as a temperature sensor for monitoring a temperature of the cardiac wall or a temperature of blood. Further sensors may also be provided on the ablation catheter. The position sensor may for example be an electromagnetically tracked position sensor, or another type of position sensor. In FIG. 1, the shaded regions in FIG. 1 represent the time of activation of each region of the heart with respect to a reference time in the cardiac cycle, and the points represent positions at which the electrical measurements are made. An EP map such as that illustrated in FIG. 1 may be generated by an EP mapping system during an EP mapping procedure.

A second intracardiac catheter is also illustrated towards the right side of each view in FIG. 1, and this represents a coronary sinus catheter. The coronary sinus catheter may be used to provide a reference position while generating the EP maps. The coronary sinus catheter illustrated in FIG. 1 may therefore include a position sensor. As with the ablation catheter 100, the coronary sinus catheter may likewise include additional sensors such as a voltage sensor and/or a current sensor and/or an impedance measurement circuit for making measurements of the cardiac wall and thereby determining a tissue state, and a temperature sensor for monitoring a temperature of the cardiac wall or a temperature of blood. Example EP mapping systems that employ sensors such as those described with reference to FIG. 1 include the KODEX-EPD cardiac imaging and mapping system under development by Philips Healthcare, USA, and the EnSite Precision™ Cardiac Mapping System, marketed by Abbott Laboratories, USA.

As may be appreciated, intracardiac sensors such as the position sensor and the electrical sensor described above with reference to intracardiac catheter 100 in FIG. 1, are susceptible to temporal motion artifacts which degrade the accuracy of their measurements. For example, cardiac motion and/or respiratory motion have the effect of degrading the accuracy of position data generated by the position sensor in FIG. 1.

By way of another example, FIG. 2 illustrates an example of temporal intracardiac sensor data 110 (Force, upper, Impedance, lower) generated by an ablation catheter, and which data includes temporal motion artifacts 120. The temporal intracardiac sensor data 110 illustrated in FIG. 2 may be generated by the ablation catheter described above with reference to FIG. 1. In FIG. 2 the upper graph represents a contact force between an intracardiac force sensor and the cardiac wall during a cardiac ablation procedure. The lower graph represents the impedance of the cardiac wall and indicates a state of tissue during the ablation procedure. The cardiac ablation begins at the Time stamp “Abl ON” and terminates at the Time stamp “Abl OFF”. The Force data illustrated in FIG. 2 may be used to confirm that the ablation probe is in contact with the cardiac wall, and thus to confirm that the Impedance data in the lower graph in FIG. 2, represents a valid measurement of the impedance of the cardiac wall. During a cardiac ablation procedure, ablation may be terminated when it is determined that the impedance of the cardiac wall has fallen by a prescribed amount. However, motion artifacts from two periodic interference signals are visible in the graphs illustrated in FIG. 2, and hamper this determination. In FIG. 2, cardiac motion artifacts are visible with a relatively shorter period of approximately 1 timestamp units, and respiratory motion artifacts are visible with a relatively longer period of approximately 4 timestamp units. As may be seen in FIG. 2, interference from these motion artifacts may even dominate smaller changes in the contact force and impedance signals, the measurement of which is desired.

The inventors have determined a method of reducing temporal motion artifacts in temporal intracardiac sensor data. The method may be used in various intracardiac systems, including the EP mapping and cardiac ablation systems described above.

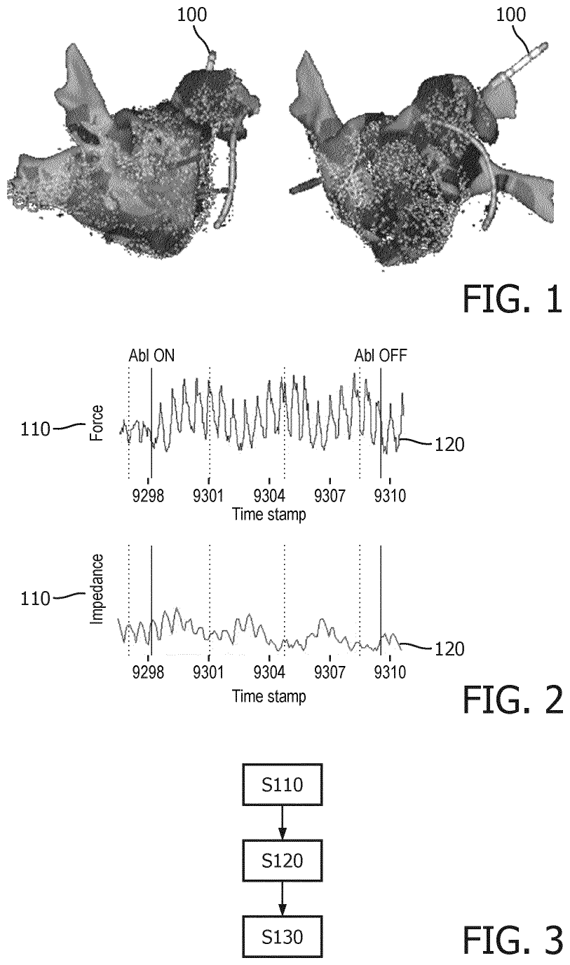

The method is described with reference to FIG. 3, which is a flowchart of an example method of reducing temporal motion artifacts in temporal intracardiac sensor data, in accordance with some aspects of the disclosure. The method may be implemented by a computer, and includes:
receiving S110 temporal intracardiac sensor data, the temporal intracardiac sensor data including temporal motion artifacts;
inputting S120 the temporal intracardiac sensor data, into a neural network trained to predict, from the temporal intracardiac sensor data, temporal motion data representing the temporal motion artifacts; and
compensating S130 for the temporal motion artifacts in the received temporal intracardiac sensor data 110 based on the predicted temporal motion data.

The temporal intracardiac sensor data received in the FIG. 3 method may be received from various sources, including an intracardiac sensor, a database, a computer readable storage medium, the cloud, and so forth. The data may be received using any form of data communication, such as wired or wireless data communication, and may be via the internet, an ethernet, or by transferring the data by means of a portable computer-readable storage medium such as a USB memory device, an optical or magnetic disk, and so forth.

In some examples the temporal intracardiac sensor data received in the FIG. 3 method may represent one or more of:
position data representing a position of one or more intracardiac position sensors;
intracardiac electrical activity data generated by one or more intracardiac electrical sensors;
contact force data representing a contact forces between a cardiac wall and one or more force sensors; and
temperature data representing a temperature of one or more intracardiac temperature sensors.

Intracardiac sensor data from other types of intracardiac sensors may be received in a similar manner. In some examples the intracardiac sensor data may be computed. For example, the sensor data may represent yaw, pitch, roll, a 3-dimensional position, or a quaternion, and this may be computed from sensors such as a gyroscope and an accelerometer to provide a position representation in terms of a model with multiple degrees of freedom. Models such as a 5 or 6-degrees of freedom “5DOF” or “6DOF” model, and so forth, are often used in conjunction with EP catheters. In a similar manner, impedance data may be computed from electrical measurements of a voltage and current by mathematically dividing the former by the latter.

In the FIG. 3 method, when the temporal intracardiac sensor data includes position data, the position data may be generated by various positioning, or “tracking” systems. By way of some examples, an example electromagnetic tracking system that uses one or more electromagnetic tracking sensors or emitters mechanically coupled to an interventional device to generate position data, is disclosed in document WO 2015/165736 A1. An example dielectric mapping tracking system that uses one or more dielectric impedance measurement circuits mechanically coupled to an interventional device to generate position data, is disclosed in document US 2019/254564 A1. An example ultrasound tracking system that uses one or more ultrasound tracking sensors or emitters mechanically coupled to an interventional device to generate position data, is disclosed in document WO 2020/030557 A1. An example fiber optical shape sensing positioning system that uses a plurality of fiber optic shape sensors mechanically coupled to an interventional device to generate position data, is disclosed in document WO2007/109778 A1. Position data from a kinematic model of a continuum robotic system may likewise be generated by sensors such as rotational and linear encoders coupled to a robotic system.

In the FIG. 3 method, when the temporal intracardiac sensor data includes intracardiac electrical activity data, the data may be generated using various electrical sensors such as a voltage, current, and charge sensors. The electrical sensors used in generating such data may include electrical contacts that are arranged to either directly, or indirectly via a dielectric layer, couple to media such as blood or cardiac tissue. Parameters such as impedance may be determined from these measurements.

When the temporal intracardiac sensor data includes contact force data and temperature data, suitable known force and temperature sensors may be used as appropriate. Other temporal intracardiac sensor data may be generated using appropriate sensors.

As illustrated by way of the example temporal intracardiac sensor data 110 in FIG. 2, the temporal intracardiac sensor data 110 includes temporal motion artifacts 120. The temporal motion artifacts 120 may include cardiac motion artifacts and/or respiratory motion artifacts. Motion artifacts from other sources may also be included in the temporal intracardiac sensor data 110.

With reference to FIG. 3, in operation S120, the temporal intracardiac sensor data 110 is inputted into a neural network 130 that is trained to predict, from the temporal intracardiac sensor data 110, temporal motion data 140, 150 representing the temporal motion artifacts 120.

In some implementations the temporal intracardiac sensor data 110 is inputted into the neural network 130 in the time domain, whereas in other implementations the temporal intracardiac sensor data 110 is inputted into the neural network 130 in the frequency domain. The temporal intracardiac sensor data 110 may be converted from the time domain to the frequency domain using a Fourier transform, or another transform, prior to inputting it into the neural network 130. In some implementations, the neural network may convert inputted temporal intracardiac sensor data 110 in the time domain to the frequency domain. Frequency domain representations such as a spectrogram, a Mel spectrogram, a wavelet representation, and so forth, may be used.

With reference to FIG. 3, in operation S130, the temporal motion artifacts 120 in the received temporal intracardiac sensor data 110 are compensated-for based on the predicted temporal motion data 140, 150. The compensation performed in operation S130 may include various techniques, and these may be carried out within, or outside, the neural network 130. The compensation may be performed in the time domain, or in the frequency domain.

In some implementations, the predicted temporal motion data 140, 150 that is predicted by the neural network 130 may have a time-domain representation. In these implementations, the compensating performed in operation S130 may include subtracting a time domain representation of the predicted temporal motion data 140, 150 from a time domain representation of the temporal intracardiac sensor data 110.

In other implementations, the temporal motion data 140, 150 that is predicted by the neural network 130 may have a frequency domain representation. In these implementations, frequencies present in this frequency domain representation of the predicted temporal motion data 140, 150, are indicative of motion artifacts. In these implementations, the compensating performed in operation S130 may include generating a mask representing motion artifact frequencies in the frequency domain representation of the predicted temporal motion data 140, 150, and multiplying the mask by a frequency domain representation of the temporal intracardiac sensor data 110. In so doing, the temporal motion artifacts 120 in the temporal intracardiac sensor data 110, may be reduced.

The result of the compensating operation S130 is temporal motion compensated intracardiac sensor data 160. The temporal motion compensated intracardiac sensor data represents the temporal intracardiac sensor data 110 with reduced temporal motion artifacts. The temporal motion compensated intracardiac sensor data 160 may, as desired, be outputted. The outputting may include outputting the temporal motion compensated intracardiac sensor data 160 in the time or the frequency domain. An inverse Fourier transform may for example be used to convert from the frequency domain to the time domain. The outputting may for example include displaying the data on a display, or storing the data to a computer-readable storage device, and so forth.

The temporal motion data 140, 150 representing the temporal motion artifacts 120, may also be outputted. This data may likewise be outputted in a time domain representation, or a frequency domain representation.

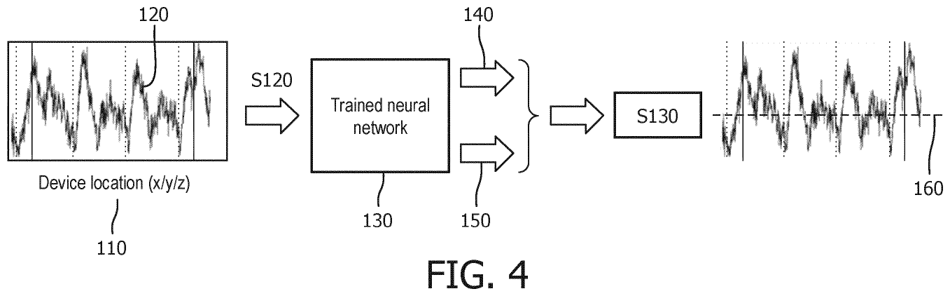

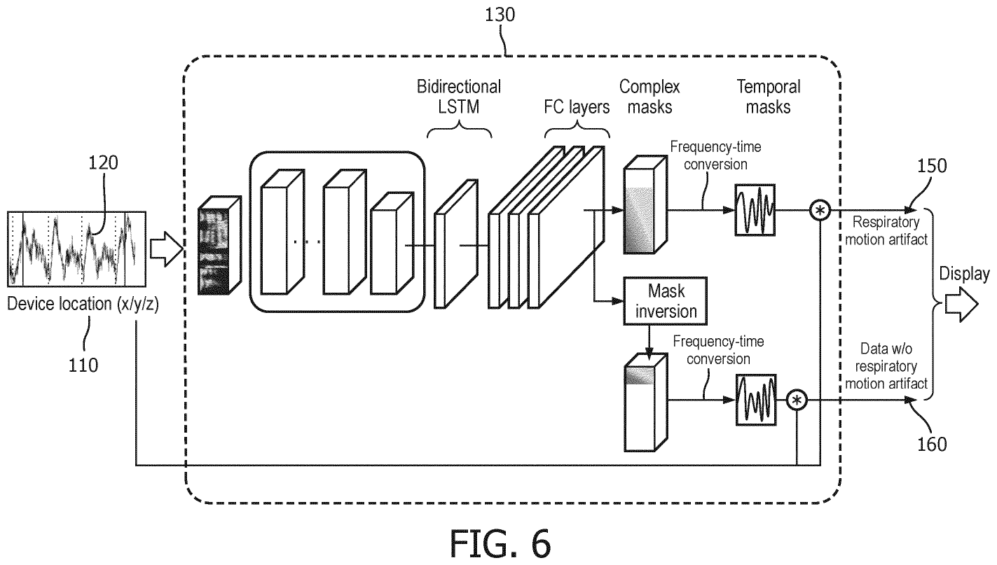

FIG. 4 is a schematic diagram illustrating an example method of reducing temporal motion artifacts in temporal intracardiac sensor data, in accordance with some aspects of the disclosure. The schematic diagram of FIG. 4 corresponds to the FIG. 3 method, and illustrates the inputting, in operation S120, of the temporal intracardiac sensor data 110, into a neural network 130. The temporal intracardiac sensor data 110 in FIG. 4 represents position data, and is labelled as “Device location x/y/z”, and includes temporal motion artifacts 120 such as cardiac motion artifacts and/or respiratory motion artifacts. The position data illustrated in FIG. 4, and also in FIG. 6, and FIGS. 8-10, represents example position data for a single dimension in a cartesian coordinate system, i.e. in an x, or y, or z dimension, and is illustrated for a single dimension in these figure for ease of illustration of the motion artifacts 120. It is however to be appreciated that position data in one, two, or more dimensions, and in cartesian, or other coordinate systems may be inputted in a similar manner.

In FIG. 4, the temporal intracardiac sensor data 110 is illustrated as being inputted in the time domain. However, this data may alternatively be inputted in the frequency domain.

The neural network 130 in FIG. 4 outputs the predicted temporal motion data 140, 150. The predicted temporal motion data 140, 150 may represent the temporal motion artifacts 120 as a temporal cardiac motion signal 140 representing the cardiac motion artifacts and/or as a temporal respiratory motion signal 150 representing the respiratory motion artifacts, respectively. In operation S130, the temporal motion artifacts 120 in the received temporal intracardiac sensor data 110 are compensated-for based on the predicted temporal motion data 140, 150.

In the example illustrated in FIG. 4, the compensating in operation S130 is performed outside the neural network 130, although the compensating may alternatively be performed by the neural network. In the example illustrated in FIG. 4, the temporal motion compensated intracardiac sensor data 160 is then outputted. As illustrated by the dashes representing intracardiac sensor data 160 in FIG. 4, in this example the temporal motion compensated intracardiac sensor data 160, or more particularly the x-component of the cartesian position data, undergoes a linear increase. The temporal motion artifacts, which can be seen as noisy semi-periodic oscillations thereupon in the inputted temporal intracardiac sensor data 110, are significantly reduced in the outputted temporal motion compensated intracardiac sensor data 160.

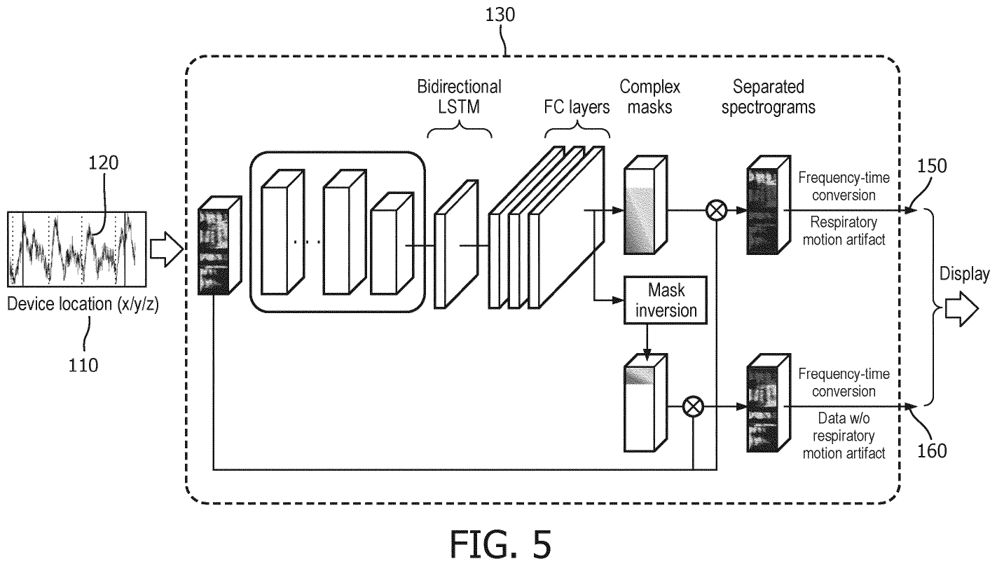

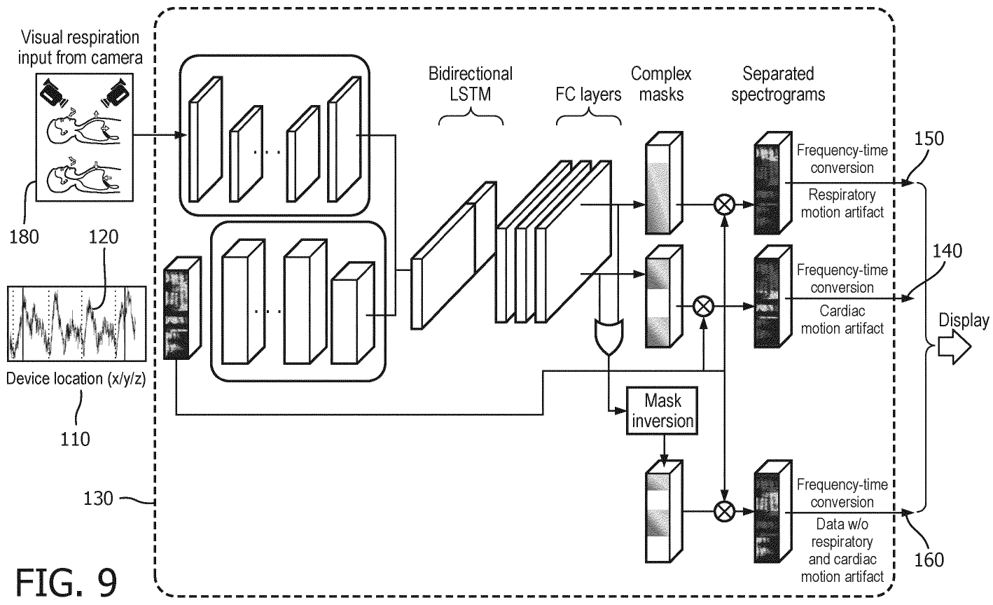

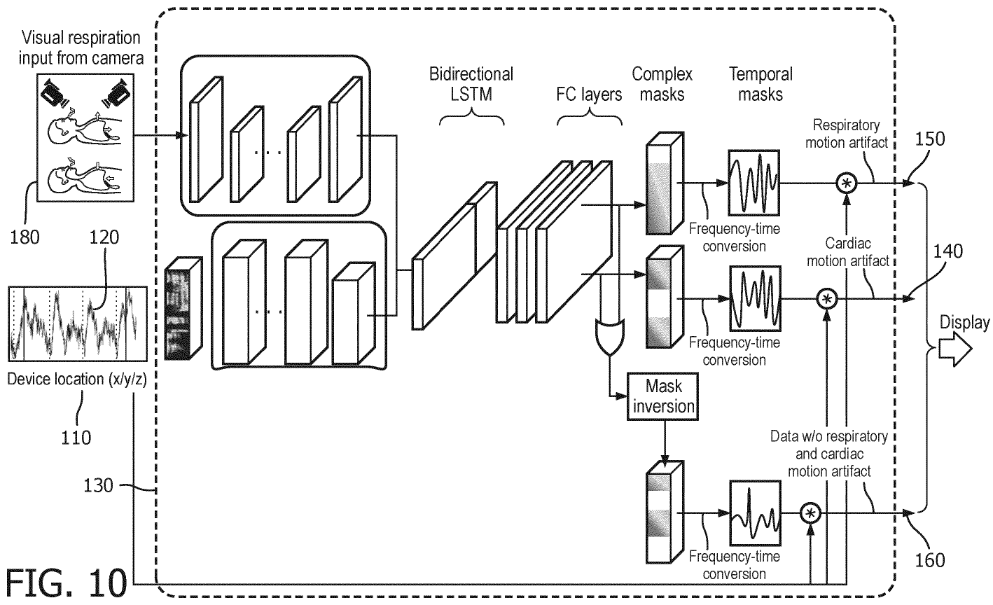

Various implementations of the neural network 130 are contemplated. These are described with reference to the neural networks illustrated in FIG. 5, FIG. 6, FIG. 9 and FIG. 10. In each of these Figures, the neural network 130 is trained to generate a frequency domain mask which is used to extract the frequencies in a spectrogram of the temporal intracardiac sensor data 110 which correspond to respiratory and/or cardiac motion artifacts. The frequencies corresponding to respiratory and/or cardiac motion artifacts are extracted by multiplying the mask by a frequency domain representation of the temporal intracardiac sensor data 110, as in FIG. 5 and FIG. 9, or the mask is converted to a time domain mask which is convolved with a time domain representation of the temporal intracardiac sensor data 110, as in FIG. 6 and FIG. 10. Elements of the neural network 130 may for example be provided by a convolutional neural network “CNN”, or by a recurrent neural network “RNN”, or by a temporal convolutional network “TCN”, or by a transformer, or by other types of neural networks.

FIG. 5 is a schematic diagram illustrating a first example of a neural network 130 for predicting temporal motion data 150 representing temporal motion artifacts, in accordance with some aspects of the disclosure. The example neural network 130 in FIG. 5 is trained to predict a temporal respiratory motion signal 150 representing respiratory motion artifacts. A temporal cardiac motion signal 140 representing cardiac motion artifacts may be predicted by the neural network 130 in a similar manner. The example neural network 130 illustrated in FIG. 5 includes convolutional layers, bi-direction long short-term memory “LSTM” layers, and fully connected “FC” layers, and may be trained in accordance with so-called weakly-labelled data using the principles disclosed in a publication by Ephrat, A. et al., entitled “Looking to listen at the cocktail party: A speaker-independent audio-visual model for speech separation,” ACM Trans. Graph., vol. 37, no. 4, 2018, doi: 10.1145/3197517.3201357.

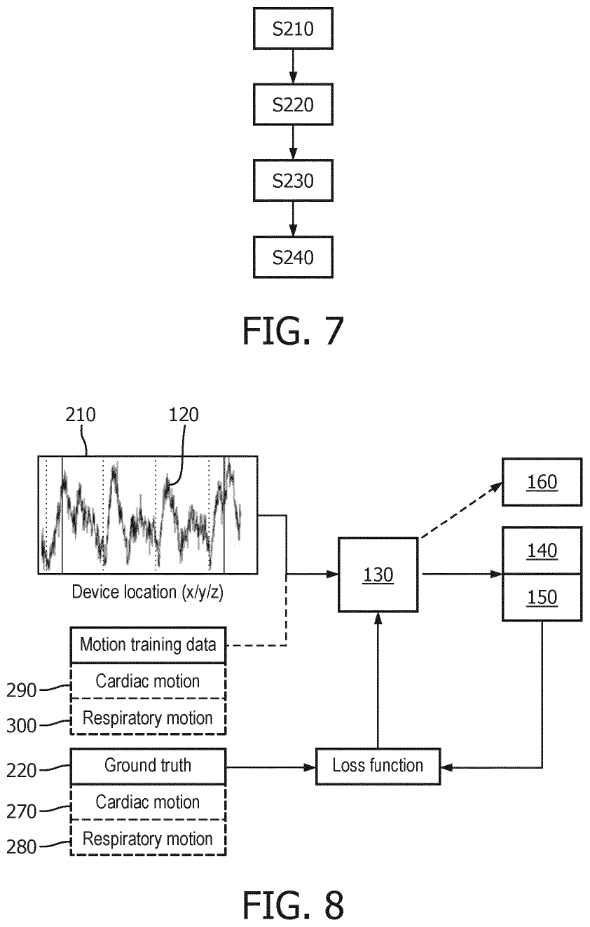

With reference to FIG. 7, which is flowchart of an example method of training a neural network to predict temporal motion data 140, 150 representing temporal motion artifacts, in accordance with some aspects of the disclosure, in some implementations, the neural network 130 is trained by:
receiving S210 temporal intracardiac sensor training data 210, the temporal intracardiac sensor training data 210 including temporal motion artifacts 120;
receiving S220 ground truth temporal motion data 220 representing the temporal motion artifacts 120; and
inputting S230 the received temporal intracardiac sensor training data 210, into the neural network 130, and adjusting S240 parameters of the neural network 130 based on a loss function representing a difference between the temporal motion data 140, 150 representing the temporal motion artifacts 120, predicted by the neural network 130, and the received ground truth temporal motion data 220 representing the temporal motion artifacts 120.

The training method is further described with reference to FIG. 8, which is a schematic diagram illustrating an example method of training a neural network to predict temporal motion data 140, 150 representing temporal motion artifacts, in accordance with some aspects of the disclosure. In more detail, with reference to FIG. 8 and the above training method, the training of neural network 130 involves the inputting of ground truth temporal motion data 220 into a loss function. The ground truth temporal motion data 220 may include ground truth cardiac motion data 270 representing cardiac motion artifacts and/or ground truth respiratory motion data 280 representing respiratory motion artifacts.

With reference to FIG. 8 and FIG. 5, the temporal intracardiac sensor training data 210 inputted to the neural network 130 in these illustrated examples is in the time domain and the neural network 130 performs a time-to-frequency conversion of the temporal intracardiac sensor training data 210 in order to generate a spectrogram that is processed by the neural network 130. In alternative implementations, the time-to-frequency conversion may take place outside the neural network, or the temporal intracardiac sensor training data 210 may be inputted in the frequency domain, and no time-to-frequency conversion is performed. If the inputted temporal intracardiac sensor training data 210 is in the time domain, the neural network 130 may compute a short-term Fourier transform, STFT, of the temporal intracardiac sensor training data 210 in order to obtain a spectrogram using a convolutional neural network, CNN, and thereby identify features associated with the temporal intracardiac sensor training data 210. The convolutions performed by the CNN are performed over the temporal axis to capture the behavior of the temporal intracardiac sensor training data 210 over time. With reference to FIG. 5, the output of the CNN is inputted into a bidirectional LSTM, BLSTM, which is a type of recurrent neural network, RNN. Other types of RNN such as a long short-term memory, LSTM, network may alternatively be used in place of the long short-term memory, LSTM network. Each intermediate layer in the neural network 130 may include, a linear convolution operation, a batch normalization, BN, dropout, nonlinearity, for example, ReLU, sigmoid, and so forth, and other operations. The output of the neural network 130 includes predicted temporal motion data 150 representing the temporal motion artifacts 120. The temporal motion data 150 predicted by the illustrated neural network includes a temporal respiratory motion signal 150 representing respiratory motion artifacts.

The FIG. 5 neural network operates in the following manner. The inputted temporal intracardiac sensor data 110 is in the time domain and a time-to-frequency conversion is applied to this data initially in order to generate a spectrogram. The neural network 130 is trained to generate a frequency domain mask which is used to extract the frequencies in the spectrogram which correspond to respiratory motion artifacts. In order to compensate for the temporal motion artifacts, this mask is multiplied by the spectrogram of the inputted temporal intracardiac sensor data 110, and the result is converted to the time domain to generate a temporal respiratory motion signal 150, which may be outputted. A mask inversion function is applied to the respiratory mask in order to create another mask which can output the residual signal, specifically the temporal motion compensated intracardiac sensor data 160. Whilst not illustrated in FIG. 5, a temporal cardiac motion signal 140, may be predicted and outputted in a similar manner.

The certainty of the outputs of the FIG. 5 neural network may be improved by inputting additional data representing e.g. cardiac motion data and/or respiratory motion data into the neural network. Such cardiac motion may for example be acquired from one or more electromagnetic position sensors that are incorporated into an intracardiac catheter. Respiratory motion data may for example be provided by an image stream generated by one or more cameras configured to image the patient's torso.

As mentioned above, the illustrated neural network 130 in FIG. 5 may additionally or alternatively predict temporal motion data in the form of a temporal cardiac motion signal 140 representing cardiac motion artifacts. These signals may be generated by training the neural network to generate one or more frequency masks “Complex masks”, which when multiplied by the inputted temporal intracardiac sensor training data 210, generate frequency domain representations of the temporal cardiac motion signal 140 and/or the temporal respiratory motion signal 150. Each mask may include a real and an imaginary channel. Time domain representations of the temporal cardiac motion signal 140 and/or the temporal respiratory motion signal 150 may be obtained by performing an inverse Fourier Transform on the frequency domain representations.

The FIG. 5 implementation performs compensation for temporal motion artifacts in the temporal intracardiac sensor data 110 in the frequency domain. In another implementation, compensation for temporal motion artifacts in the temporal intracardiac sensor data 110 may be performed in the time domain. Thereto, FIG. 6 is a schematic diagram illustrating a second example of a neural network 130 for predicting temporal motion data 140, 150 representing temporal motion artifacts, in accordance with some aspects of the disclosure. Items in FIG. 6 that are labelled with the same label as in FIG. 5 refer to the same item.

As in FIG. 5, the temporal intracardiac sensor data 110 that is inputted to the neural network 130 in FIG. 6 is initially converted from the temporal domain to the frequency domain to provide a spectrogram before being processed further by the neural network. In alternative examples, the time-to-frequency conversion may take place outside the neural network 130, or the temporal intracardiac sensor data 110 may be inputted in the frequency domain and no time-to-frequency conversion performed. In the FIG. 6 implementation, the neural network is trained to generate a frequency domain mask which is used to extract the frequencies in the spectrogram which correspond to respiratory motion artifacts. This mask is converted to a time domain mask, and the result is convolved with the inputted time domain temporal intracardiac sensor data 110 to generate a temporal respiratory motion signal 150, which may be outputted. A mask inversion function is applied to the respiratory mask in order to create a further mask, which can then be converted to a time domain mask and convolved with the temporal intracardiac sensor data 110 in order to generate a residual signal, specifically the temporal motion compensated intracardiac sensor data 160. Whilst not illustrated in FIG. 5, a temporal cardiac motion signal 140, may be predicted and outputted in a similar manner.

As mentioned in relation to FIG. 5, the certainty of the outputs of the neural network 130 in FIG. 6 may be improved by inputting additional data representing e.g. cardiac motion data and/or respiratory motion data, into the neural network. Such cardiac motion may for example be acquired from one or more electromagnetic position sensors that are incorporated into an intracardiac catheter. Respiratory motion data may for example be provided by an image stream generated by one or more cameras configured to image the patient's torso.

Returning to FIG. 8, during training the neural network 130 in FIG. 5 and FIG. 6, the time, or frequency, domain representations of the temporal cardiac motion signal 140 and/or the temporal respiratory motion signal 150 are inputted to a loss function, and the value of the loss function used as feedback to adjust the weights and biases, i.e. “parameters” of the neural network 130. The loss function computes a difference between the temporal motion data 140, 150, predicted by the neural network 130, and the received ground truth temporal motion data 220.

The value of the loss function may be computed using functions such as the negative log-likelihood loss, the L2 loss, or the Huber loss, or the cross entropy loss, and so forth. During training, the value of the loss function is typically minimized, and training is terminated when the value of the loss function satisfies a stopping criterion. Sometimes, training is terminated when the value of the loss function satisfies one or more of multiple criteria.

Various methods are known for solving the loss minimization problem such as gradient descent, Quasi-Newton methods, and so forth. Various algorithms have been developed to implement these methods and their variants including but not limited to Stochastic Gradient Descent “SGD”, batch gradient descent, mini-batch gradient descent, Gauss-Newton, Levenberg Marquardt, Momentum, Adam, Nadam, Adagrad, Adadelta, RMSProp, and Adamax “optimizers” These algorithms compute the derivative of the loss function with respect to the model parameters using the chain rule. This process is called backpropagation since derivatives are computed starting at the last layer or output layer, moving toward the first layer or input layer. These derivatives inform the algorithm how the model parameters must be adjusted in order to minimize the error function. That is, adjustments to model parameters are made starting from the output layer and working backwards in the network until the input layer is reached. In a first training iteration, the initial weights and biases are often randomized. The neural network then predicts the output data, which is likewise, random. Backpropagation is then used to adjust the weights and the biases. The training process is performed iteratively by making adjustments to the weights and biases in each iteration. Training is terminated when the error, or difference between the predicted output data and the expected output data, is within an acceptable range for the training data, or for some validation data. Subsequently the neural network may be deployed, and the trained neural network makes predictions on new input data using the trained values of its parameters. If the training process was successful, the trained neural network accurately predicts the expected output data from the new input data.

The temporal intracardiac sensor training data 210 that is inputted to the neural network 130 during training may be provided by data measured on a subject, or by simulated data. The temporal motion artifacts 120 in the measured data are inherent. Simulated training data 210 with temporal motion artifacts may be provided by summing motion artifact-free sensor data with signals representing motion from e.g. cardiac and/or respiratory motion.

The ground truth cardiac motion data 270 representing cardiac motion artifacts, and the ground truth respiratory motion data 280 may originate from various sources. The ground truth cardiac motion data 270 that is used to train the neural network 130, may for example be provided by:
an intracardiac probe configured to detect intracardiac activation signals; or
an extra-corporeal electrocardiogram sensor; or
one or more cameras configured to detect blood-flow-induced changes in skin color; or
a transthoracic ultrasound echocardiography, TTE, imaging system; or
a transesophageal ultrasound echocardiography, TEE, imaging system; or a microphone. The microphone may be intra-corporeal, for example arranged to be disposed within the cardiac region, or extra-corporeal.

The ground truth respiratory motion data 280 that is used to train the neural network 130, may for example be provided by:
one or more extra-corporeal impedance measurement circuits configured to measure a conductivity of a chest or abdominal cavity of a subject; or
one or more cameras configured to image a chest or abdominal cavity of a subject; or
an impedance band mechanically coupled to a chest or abdominal cavity of a subject; or
a mechanical ventilation assistance device coupled to a subject; or
a position sensing system configured to detect the position of one or more extra-corporeal markers disposed on a chest or abdominal cavity of a subject.

The one or more cameras may include a monocular camera or a stereo camera, which may be an RGB, a grayscale, a hyperspectral, a time-of-flight, or an infrared camera, arranged to view a torso of a subject. The one or more cameras may include an image processing controller configured to extract the respiration pattern from acquired image frames generated by the one or more camera. The impedance band may include an elastic strap that encircles the torso or abdominal cavity of a subject. The impedance band converts the expansion and contraction of the rib cage or abdominal cavity into respiration waveforms using a signal processing module. The extra-corporeal markers may include optical markers, such as retroreflective skin-mounted markers, or electromagnetic coils, the positions of which may be respectively measured by a stereotactic optical navigation systems, and an electromagnetic tracking system.

FIG. 9 is a schematic diagram illustrating a third example of a neural network 130 for predicting temporal motion data 140, 150 representing temporal motion artifacts, in accordance with some aspects of the disclosure. The example neural network 130 in FIG. 9 is trained to predict a temporal respiratory motion signal 150 representing respiratory motion artifacts. The neural network 130 illustrated in FIG. 9 corresponds to the neural network in FIG. 5, and is likewise trained to predict a temporal respiratory motion signal 150 representing respiratory motion artifacts. As in FIG. 5, the neural network 130 in FIG. 9 is trained to predict the temporal respiratory motion signal 150 from the inputted temporal intracardiac sensor data 110. The neural network 130 in FIG. 9 is also trained to predict the temporal respiratory motion signal 150 from inputted respiratory motion data 180. A temporal cardiac motion signal 140 representing cardiac motion artifacts is also predicted by the neural network 130 in a similar manner.

The neural network in FIG. 9 operates in the same manner as described above in relation to FIG. 5, and additionally includes a convolutional neural network for processing the inputted respiratory motion data 180. During training, this CNN learns a feature representation of the respiratory motion. The convolutions performed in in this CNN are performed over the temporal axis to capture the behavior of the respiratory motion over time. With reference to FIG. 9, these representations are then concatenated and inputted to the bidirectional LSTM, BLSTM, that was described above with reference to FIG. 5. Thus, in contrast to the FIG. 5 neural network 130, the neural network illustrated in FIG. 9 additionally uses the inputted respiratory motion data 180 to predict the temporal respiratory motion signal 150. The use of additional inputted respiratory motion data 180 fine-tunes the accuracy of the neural network's predictions.

In contrast to the neural network illustrated in FIG. 5, in FIG. 9, respiratory motion data 180, is also inputted to the neural network 130 as well as the intracardiac sensor data 110. The neural network 130 is trained to generate a frequency domain mask which is used to extract frequencies in the spectrogram which correspond to respiratory motion artifacts. A combination of two masks is inverted and multiplied by the spectrogram of the inputted temporal intracardiac sensor data 110 to generate the spectrogram of the intracardiac sensor data with reduced respiratory motion artifacts and with reduced cardiac motion artifacts. This spectrogram may then be converted to the time domain to provide the temporal motion compensated intracardiac sensor data 160. In a similar manner, cardiac motion data 170, not illustrated in FIG. 9, may be inputted into the neural network 130 in addition to, or instead of, the respiratory motion data 180 in order to compensate for cardiac motion. In a similar manner to that described above in relation to FIG. 5 for the temporal respiratory motion signal 150, the illustrated neural network 130 in FIG. 9 also predicts a temporal cardiac motion signal 140 representing cardiac motion artifacts. The predicted temporal cardiac motion signal 140 and/or temporal respiratory motion signal 150, may be additionally or alternatively predicted based on inputted cardiac motion data 170. The inputted cardiac motion data 170 may be processed by a convolutional neural network, in other words, in a similar manner to the inputted respiratory motion data 180.

The FIG. 9 implementation performs compensation for temporal motion artifacts in the temporal intracardiac sensor data 110 in the frequency domain. In another implementation, compensation for temporal motion artifacts in the temporal intracardiac sensor data 110 is performed in the time domain. Thereto, FIG. 10 is a schematic diagram illustrating a fourth example of a neural network 130 for predicting temporal motion data 140, 150 representing temporal motion artifacts, in accordance with some aspects of the disclosure. Items in FIG. 10 that are labelled with the same label as in FIG. 9 refer to the same item. As illustrated in FIG. 9, in FIG. 10, respiratory motion data 180 is inputted into the neural network 130 as well as the intracardiac sensor data 110. The neural network in FIG. 10 processes the spectrogram of the intracardiac sensor data 110 as well as the respiratory motion data. The neural network 130 is trained to generate frequency domain masks which are converted to time domain masks that extract respiratory motion artifacts and cardiac motion artifacts by being convolved with the inputted time-domain intracardiac sensor data 110. A combination of two masks is inverted in FIG. 10 and converted to a time domain mask that is convolved with the intracardiac sensor data 110 to generate intracardiac sensor data without respiratory motion artifacts or cardiac motion artifacts, This data may then be converted to the time domain, specifically to provide the temporal motion compensated intracardiac sensor data 160, and outputted.

The cardiac motion data 170 and respiratory motion data 180 may be provided by any of the sources that were described above for the ground truth cardiac motion data 270, and the ground truth respiratory motion data 280, respectively. For example, the cardiac motion data 170 may for example be provided by an intracardiac probe configured to detect intracardiac activation signals, and the respiratory motion data 180 may for example be provided by one or more extra-corporeal impedance measurement circuits configured to measure a conductivity of a chest or abdominal cavity of a subject.

During inference with the FIG. 9 neural network, various additional operations may be performed as compared to those described in FIG. 3 in relation to FIG. 5. During inference with the FIG. 9 implementation, the method additionally may include:
converting the received temporal intracardiac sensor data 110 to a frequency domain representation; and wherein the temporal motion artifacts 120 comprise cardiac motion artifacts and/or respiratory motion artifacts; and wherein the neural network 130 is trained to predict, from the temporal intracardiac sensor data 110, the temporal motion data 140, 150 representing the temporal motion artifacts 120 as a temporal cardiac motion signal 140 representing the cardiac motion artifacts and/or as a temporal respiratory motion signal 150 representing the respiratory motion artifacts, respectively; and wherein the temporal cardiac motion signal 140 and/or the temporal respiratory motion signal 150 comprise a temporal variation of a frequency domain representation of said data;
and wherein the compensating S130 is performed by masking the frequency domain representation of the received temporal intracardiac sensor data 110 with the frequency domain representation of the temporal cardiac motion signal 140 and/or the frequency domain representation of the temporal respiratory motion signal 150.

The training of the neural network 130 illustrated in FIG. 9 likewise includes additional operations to those described above with reference to FIG. 7 in relation to the neural network 130 in FIG. 5. In the FIG. 9 neural network 130, the temporal motion data 140, 150 predicted by the neural network comprises a temporal cardiac motion signal 140 representing cardiac motion artifacts and/or a temporal respiratory motion signal 150 representing respiratory motion artifacts, and the ground truth temporal motion data 220 representing the temporal motion artifacts 120 comprises ground truth cardiac motion data 270 and/or ground truth respiratory motion data 280 respectively representing the cardiac motion artifacts and the respiratory motion artifacts, and the neural network 130 is trained to predict the cardiac motion signal 140 and/or the temporal respiratory motion signal 150 from the temporal intracardiac sensor data 110, and from cardiac motion data 170 and/or respiratory motion data 180 corresponding to the temporal motion artifacts 120. As compared to the FIG. 5 neural network, training of the FIG. 9 neural network 130 also includes:
inputting cardiac motion training data 290 corresponding to the cardiac motion data 170 and/or respiratory motion training data 300 corresponding to the respiratory motion data 180 into the neural network 130;
and wherein the loss function is based on a difference between the temporal cardiac motion signal 140 and/or the temporal respiratory motion signal 150, predicted by the neural network 130, and the received ground truth cardiac motion data 270 and/or the ground truth respiratory motion data 280, respectively.

The training of the FIG. 9 neural network may also be performed in the same manner as described in FIG. 8. Differences in training of the FIG. 9 neural network as compared to training the FIG. 5 neural network are illustrated by way of the dashed arrows between the motion training data 290, 300 and the neural network 130. The dashed arrows indicate that the training of the FIG. 9 neural network also includes inputting the ground truth temporal motion data 220 into the loss function, and that motion training data such as cardiac motion training data 290 and/or respiratory motion training data 300, are inputted into the neural network 130.

Additional input data to the neural network 130 described above in FIG. 5 and FIG. 9 may also be provided and used during inference and/or training in order to further improve the accuracy of its predictions. For example, the neural network may be inputted with position data that indicates an origin within the heart of the received temporal intracardiac sensor data 110. This may for example be used by the neural network to fine-tune its predictions of the temporal motion data 140, 150 to those expected in a particular cardiac region. This additional input data to the neural network may likewise include, for example, information relating to a medical condition such as arrythmia, the heart chamber of the arrythmia, whether the temporal intracardiac sensor data corresponds to a position in a blood pool, whether the temporal intracardiac sensor data corresponds to a position in contact with tissue, the type of arrythmia, and so forth.

In any of the methods described above, an estimated certainty of the temporal motion data 140, 150 predicted by the neural network 130, may be computed. The estimated certainty may be based on one of more of the following:
a difference between predicted output 140, 150, and the ground truth temporal motion data 220 inputted during training;
a standard deviation of the predicted output 140, 150. For instance a high standard deviation may indicate low certainty in the predicted output 140, 150 since, for example, the inputted temporal intracardiac sensor data 110 has a light level of interference; a quality of camera images used to determine the cardiac motion data 170 and respiratory motion data 180. For example, if a subject's skin is not clearly visible in the image, then the certainty in the output of the neural network may be low since the cardiac signal may be inaccurate.
dropout as a Bayesian approximation, wherein the outputs of a plurality of neurons in the neural network are ignored, and inference is performed on input data several times and a mean and standard deviation of the outputs are computed. High standard deviation may indicate a low certainty in the predicted output, whereas low standard deviation may indicate a high certainty in the predicted output.

A system for reducing temporal motion artifacts from temporal intracardiac sensor data is also provided in accordance with the present disclosure. The system includes one or more processors configured to perform one or more elements of the methods described above.

A method of training the above-mentioned neural networks, is also provided. Thereto, a computer-implemented method of providing a neural network for predicting temporal motion data representing temporal motion artifacts from temporal intracardiac sensor data, includes:
receiving S210 temporal intracardiac sensor training data 210, the temporal intracardiac sensor training data 210 including temporal motion artifacts 120;
receiving S220 ground truth temporal motion data 220 representing the temporal motion artifacts 120;
inputting S230 the received temporal intracardiac sensor training data 210, into a neural network 130, and adjusting S240 parameters of the neural network 130 based on a loss function representing a difference between temporal motion data 140, 150 representing the temporal motion artifacts 120, predicted by the neural network 130, and the received ground truth temporal motion data 220 representing the temporal motion artifacts 120.

The training method may incorporate one or more operations described above in relation to the trained neural network 130. For example, the ground truth temporal motion data 220 representing the temporal motion artifacts 120 may include ground truth cardiac motion data 270 representing cardiac motion artifacts and/or ground truth respiratory motion data 280 representing respiratory motion artifacts.

By way of an example, during training, the temporal motion data 140, 150 predicted by the neural network may comprise a temporal cardiac motion signal 140 representing cardiac motion artifacts and/or a temporal respiratory motion signal 150 representing respiratory motion artifacts, and the ground truth temporal motion data 220 representing the temporal motion artifacts 120 may comprise ground truth cardiac motion data 270 and/or ground truth respiratory motion data 280 respectively representing the cardiac motion artifacts and the respiratory motion artifacts, and the neural network 130 is trained to predict the cardiac motion signal 140 and/or the temporal respiratory motion signal 150 from the temporal intracardiac sensor data 110, and from cardiac motion data 170 and/or respiratory motion data 180 corresponding to the temporal motion artifacts 120;

In some examples, motion training data 290, 300 may also be inputted into the neural network 130 during training. In these examples, the method may further include: inputting cardiac motion training data 290 corresponding to the cardiac motion data 170 and/or respiratory motion training data 300 corresponding to the respiratory motion data 180 into the neural network 130;
and wherein the loss function is based on a difference between the temporal cardiac motion signal 140 and/or the temporal respiratory motion signal 150, predicted by the neural network 130, and the received ground truth cardiac motion data 270 and/or the ground truth respiratory motion data 280, respectively.

In another example, a processing arrangement for providing a neural network for predicting temporal motion data representing temporal motion artifacts from temporal intracardiac sensor data, is provided. The processing arrangement includes one or more processors configured to perform the above-described training method.

The above examples are to be understood as illustrative of the present disclosure and not restrictive. Further examples are also contemplated. For instance, the examples described in relation to the computer-implemented method, may also be provided by a computer program product, or by a computer-readable storage medium, or by a processing arrangement, or by a system, in a corresponding manner. It is to be understood that a feature described in relation to any one example may be used alone, or in combination with other described features, and may also be used in combination with one or more features of another of the examples, or a combination of other examples. Furthermore, equivalents and modifications not described above may also be employed without departing from the scope of the invention, which is defined in the accompanying claims. In the claims, the word “comprising” does not exclude other elements or operations, and the indefinite article “a” or “an” does not exclude a plurality. The mere fact that certain features are recited in mutually different dependent claims does not indicate that a combination of these features cannot be used to advantage. Any reference signs in the claims should not be construed as limiting their scope.

1. A computer-implemented method of reducing temporal motion artifacts in temporal intracardiac sensor data, the method comprising:
receiving temporal intracardiac sensor data including temporal motion artifacts;
predicting, from the temporal intracardiac sensor data, temporal motion data representing the temporal motion artifacts; and
compensating for the temporal motion artifacts in the received temporal intracardiac sensor data based on the predicted temporal motion data.

2. The computer-implemented method according to claim 1,
wherein the temporal motion artifacts comprise at least one of cardiac motion artifacts and respiratory motion artifacts; and
wherein the temporal motion data represents the temporal motion artifacts as at least one of a temporal cardiac motion signal representing the cardiac motion artifacts and a temporal respiratory motion signal representing the respiratory motion artifacts.

3. The computer-implemented method according to claim 2, further comprising:
converting the received temporal intracardiac sensor data to a frequency domain representation, wherein at least one of the temporal cardiac motion signal and the temporal respiratory motion signal comprise a temporal variation of a frequency domain representation of the data; and
masking the frequency domain representation of the received temporal intracardiac sensor data with at least one of the frequency domain representation of the temporal cardiac motion signal and the frequency domain representation of the temporal respiratory motion signal to compensate the temporal intracardiac sensor data.

4. The computer-implemented method according to claim 1, further comprising:
outputting the temporal motion compensated intracardiac sensor data.

5. The computer-implemented method according to claim 1, wherein the temporal intracardiac sensor data represents one or more of:
position data representing a position of one or more intracardiac position sensors;
intracardiac electrical activity data generated by one or more intracardiac electrical sensors;
contact force data representing a contact forces between a cardiac wall and one or more force sensors; and
temperature data representing a temperature of one or more intracardiac temperature sensors.

6. The computer-implemented method according to claim 1,
wherein the temporal motion data is predicted, from the temporal intracardiac sensor data, by a neural network trained by:
receiving temporal intracardiac sensor training data, the temporal intracardiac sensor training data including temporal motion artifacts;
receiving ground truth temporal motion data representing the temporal motion artifacts; and
inputting the received temporal intracardiac sensor training data, into the neural network, and adjusting parameters of the neural network based on a loss function representing a difference between the temporal motion data representing the temporal motion artifacts, predicted by the neural network, and the received ground truth temporal motion data representing the temporal motion artifacts.

7. The computer-implemented method according to claim 6, wherein the temporal motion data predicted by the neural network comprises a temporal cardiac motion signal representing cardiac motion artifacts, and wherein the ground truth temporal motion data representing the temporal motion artifacts comprises ground truth cardiac motion data representing the cardiac motion artifacts, and wherein the neural network is trained to predict the cardiac motion signal from the temporal intracardiac sensor data, and from cardiac motion data;
and wherein the neural network is trained by further:
inputting cardiac motion training data corresponding to the cardiac motion data into the neural network;
and wherein the loss function is based on a difference between the temporal cardiac motion signal predicted by the neural network, and the received ground truth cardiac motion data.

8. The computer-implemented method according to claim 6,
wherein the temporal motion data predicted by the neural network comprises a temporal respiratory motion signal representing respiratory motion artifacts, and wherein the ground truth temporal motion data representing the temporal motion artifacts comprises ground truth respiratory motion data representing the respiratory motion artifacts, and wherein the neural network is trained to predict the temporal respiratory motion signal from the temporal intracardiac sensor data, and from respiratory motion data corresponding to the temporal motion artifacts;
and wherein the neural network is trained by further:
inputting respiratory motion training data corresponding to the respiratory motion data into the neural network;
and wherein the loss function is based on a difference between the temporal respiratory motion signal, predicted by the neural network, and the received ground truth respiratory motion data.

9. The computer-implemented method according to claim 7, wherein the cardiac motion data is provided by:
an intracardiac probe configured to detect intracardiac activation signals;
an extra-corporeal electrocardiogram sensor;
one or more cameras configured to detect blood-flow-induced changes in skin color;
a transthoracic ultrasound echocardiography (TTE) imaging system;
a transesophageal ultrasound echocardiography (TEE) imaging system; or
a microphone.

10. The computer-implemented method according to claim 8, wherein the respiratory motion data is provided by:
one or more extra-corporeal impedance measurement circuits configured to measure a conductivity of a chest or abdominal cavity of a subject;
one or more cameras configured to image a chest or abdominal cavity of a subject;
an impedance band mechanically coupled to a chest or abdominal cavity of a subject;
a mechanical ventilation assistance device coupled to the subject; or
a position sensing system configured to detect the position of one or more extra-corporeal markers disposed on of a chest or abdominal cavity of a subject.

11. The computer-implemented method according to claim 1, further comprising:
converting at least one of the received temporal intracardiac sensor data, and/or the received temporal intracardiac sensor training data, and/or the received cardiac motion data and respiratory motion data to a frequency domain representation prior to the inputting of the data into the neural network; and converting the received ground truth temporal motion data to a frequency domain representation prior to computing the loss function.

12. The computer-implemented method according to claim 1, further comprising:
computing an estimated certainty of the predicted temporal motion data representing the temporal motion artifacts.

13. The computer-implemented method according to claim 1, further comprising providing a neural network for predicting temporal motion data representing temporal motion artifacts from temporal intracardiac sensor data by:
receiving temporal intracardiac sensor training data, the temporal intracardiac sensor training data including temporal motion artifacts;
receiving ground truth temporal motion data representing the temporal motion artifacts;
inputting the received temporal intracardiac sensor training data, into a neural network, and adjusting parameters of the neural network based on a loss function representing a difference between temporal motion data representing the temporal motion artifacts, predicted by the neural network, and the received ground truth temporal motion data representing the temporal motion artifacts.

14. A system of reducing temporal motion artifacts in temporal intracardiac sensor data, the system comprising
a processor communicatively coupled to memory, the processor configured to:
receive temporal intracardiac sensor data including temporal motion artifacts;
predict, from the temporal intracardiac sensor data, temporal motion data representing the temporal motion artifacts; and
compensate for the temporal motion artifacts in the received temporal intracardiac sensor data based on the predicted temporal motion data.

15. A non-transitory computer-readable storage medium having stored a computer program comprising instructions which when executed by a processor, cause the processor to:
receive temporal intracardiac sensor data including temporal motion artifacts;
predict, from the temporal intracardiac sensor data, temporal motion data representing the temporal motion artifacts; and
compensate for the temporal motion artifacts in the received temporal intracardiac sensor data based on the predicted temporal motion data.

16. The system according to claim 14, wherein the processor is further configured to apply a machine-learning model trained to predict the temporal motion data from the temporal intracardiac sensor data.

17. The non-transitory computer-readable storage medium according to claim 15, wherein, when executed the processor, the instructions further cause the processor to apply a machine-learning model trained to predict the temporal motion data from the temporal intracardiac sensor data.
